(19) United States
(12) Patent Application Publication
(10) Pub. No.: US 2005/0183212 A1
(43) Pub. Date: Aug. 25, 2005

(54) Compositions for dyeing keratin fibers, comprising at least one alcohol oxidase and at least one direct dye comprising an acid functional group, or a salt thereof, and processes using these compositions

(76) Inventor: Gregory Plos, Tokyo (JP)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/044,182

(22) Filed: Jan. 28, 2005

Related U.S. Application Data
(60) Provisional application No. 60/545,941, filed on Feb. 20, 2004.

(30) Foreign Application Priority Data
Jan. 28, 2004 (FR) 04 00784

(51) Int. Cl. A61K 7/13

(52) U.S. Cl. 8/406

(57) ABSTRACT

The present disclosure relates to compositions for dyeing keratin fibers, for example human keratin fibers such as the hair, comprising, in a medium that is suitable for dyeing, at least one oxidation dye precursor, at least one alcohol oxidase enzyme, at least one substrate for the enzyme, and at least one non-quinone direct dye comprising an acid functional group. This disclosure also relates to processes for dyeing keratin fibers, which comprise applying the compositions disclosed herein, and to dyeing kits comprising the compositions as disclosed herein.

This application claims benefit of U.S. Provisional Application No. 60/545,941, filed Feb. 20, 2004.

The present disclosure relates to compositions for dyeing keratin fibers, for example human keratin fibers, such as the hair, comprising, in a medium that is suitable for dyeing, at least one oxidation dye precursor, at least one alcohol oxidase enzyme, at least one substrate for the enzyme, and at least one direct dye comprising an acid functional group, or a salt thereof.

It is known practice to dye keratin fibers, such as human hair, with dye compositions containing oxidation dye precursors, for example ortho- or para-phenylenediamines, ortho- or para-aminophenols, and heterocyclic compounds, which are generally referred to as oxidation bases. These oxidation bases are colorless or weakly colored compounds which, when combined with oxidizing products, may give rise to colored compounds by a process of oxidative condensation.

It is also known that the shades obtained with these oxidation bases may be varied by combining them with one or more couplers or coloration modifiers, the latter being chosen, for example, from aromatic meta-diamines, meta-aminophenols, meta-diphenols, and certain heterocyclic compounds such as indole compounds.

The variety of molecules used as oxidation bases and couplers makes it possible to obtain a wide range of colors.

The “permanent” coloration obtained by means of these oxidation dyes should generally satisfy a certain number of requirements. First, it should have no toxicological drawbacks. Second, it should allow shades of the desired intensity to be obtained. Third, it should have good resistance to external agents such as light, bad weather, washing, permanent waving, perspiration, and rubbing.

The dyes should also allow white keratin fibers to be covered. They should also be as unselective as possible, that is to say that they should allow the smallest possible differences in coloration to be produced over the entire length of the same keratin fiber, which is generally differently sensitized (i.e. damaged) between its end and its root.

Dyeing is generally performed in strongly alkaline medium, in the presence of hydrogen peroxide. However, the use of alkaline media in the presence of hydrogen peroxide can have the drawbacks of causing considerable degradation of the fibers, and also bleaching of keratin fibers, which is not always desirable.

Furthermore, this type of composition can have the drawback of having to prepare a mixture of the aqueous hydrogen peroxide solution and the dye support at the time of application of the composition to the keratin fibers.

Oxidation dyeing of keratin fibers may also be performed using oxidizing systems other than hydrogen peroxide, such as enzymatic systems comprising, for example, enzymes of the 2-electron oxidase type. Thus, French Patent Application FR 2,769,219 describes the use of a uricase enzyme and of its uric acid substrate in oxidation dyeing to dye keratin fibers. These enzymes catalyze the oxidation of a substrate via atmospheric oxygenation to generate at least one oxidation product, and also hydrogen peroxide. The hydrogen peroxide generated may be used to oxidize oxidation dye precursors and, consequently, to produce the color on the hair. This system can make it possible to perform oxidation dyeing without mixing at the time of use. However, although the dye formulations using alcohol oxidase can be used under conditions that do not cause degradation of the hair comparable to that generated by the formulations using aqueous hydrogen peroxide solution, and although they offer the possibility of being formulated all-in-one, they can lead to colorations that are still insufficient with regard to the homogeneity of the color, the dyeing power, and the chromaticity.

European Patent Application EP-A-0,310,675 describes the use of oxidation dye precursors of benzenic type in combination with enzymes such as pyranose oxidase and glucose oxidase. The compositions described in this patent application may also comprise direct dyes. However, the colorations obtained using these compositions can be unsatisfactory.

The aim of the present disclosure is to provide novel compositions for dyeing keratin fibers by oxidation dyeing, which respect the nature of the keratin fiber, which can offer the possibility of being formulated all-in-one, and which can lead to homogeneous, powerful colors and strong chromaticity.

The Inventor has discovered novel compositions comprising at least one oxidation dye precursor, at least one enzyme of alcohol oxidase type, at least one substrate for the enzyme, and at least one direct dye, other than a quinone direct dye, comprising an acid functional group, or a salt thereof. The compositions according to the present disclosure can produce dyeing results with chromatic, strong, unselective, and resistant colors, and are capable of generating varied shades of intense and uniform color, without any significant degradation of the keratin fibers.

Other characteristics, aspects, subjects and advantages of the present disclosure will become evident upon reading the description and the examples that follow.

The term “direct dye comprising an acid functional group, or a salt thereof,” as used herein, means a direct dye bearing an organic acid functional group, such as a carboxylic, sulfonic or phosphoric acid functional group, or a salt thereof with alkali metals or alkaline-earth metals, with the exception of quinone direct dyes.

The at least one direct dye comprising an acid functional group, or a salt thereof, that may be used according to the present disclosure, includes, but is not limited to, acidic nitro direct dyes, acidic azo dyes, acidic azine dyes, acidic triarylmethane dyes, acidic indoamine dyes, and non-quinone acidic natural dyes. As a non-limiting example, the following dyes may be used:

| | |
|---|---|
| (C.I. 10316) | Sodium salt of 2,4-dinitro-1-naphthol-7-sulfonic acid |
| (C.I. 10383) | Acid Orange 3 |
| (C.I. 13015) | Acid Yellow 9/Food Yellow 2 |
| (C.I. 14780) | Direct Red 45/Food Red 13 |
| (C.I. 13711) | Acid Black 52 |
| (C.I. 13065) | Acid Yellow 36 |
| (C.I. 14700) | Sodium salt of 1-hydroxy-2-(2′,4′-xylyl-5- |
| | sulfonatoazo)naphthalene-4-sulfonic acid/Food Red 1 |
| (C.I. 14720) | Acid Red 14/Food Red 3/Mordant Blue 79 |
| (C.I. 14805) | Acid Brown 4 |
| (C.I. 15510) | Acid Orange 7/Pigment Orange 17/Solvent Orange 49 |
| (C.I. 15985) | Food Yellow 3/Pigment Yellow 104 |
| (C.I. 16185) | Acid Red 27/Food Red 9 |
| (C.I. 16230) | Acid Orange 10/Food Orange 4 |
| (C.I. 16250) | Acid Red 44 |
| (C.I. 17200) | Acid Red 33/Food Red 12 |
| (C.I. 13683) | Sodium salt of 1-(3′-nitro-5′-sulfo-6′-oxophenylazo)-8- |
| | acetamidonaphthalene-3,6-disulfonic acid/Food Red 11 |
| (C.I. 18065) | Sodium salt of 1-hydroxy-2-(2′-methylphenylazo)-8- |
| | acetamidonaphthalene-3,6-disulfonic acid/Acid Red 35 |
| (C.I. 19125) | Acid Violet 3 |
| (C.I. 18055) | Acid Red 35 |
| (C.I. 18055) | Acid Violet 7 |
| (C.I. 18130) | Acid Red 135 |
| (C.I. 19130) | Acid Yellow 27 |
| (C.I. 19140) | Acid Yellow 23/Food Yellow 4 |
| (C.I. 20170) | 4′-(Sulfonato-2″,4″-dimethyl)bis(2,6-phenylazo)-1,3- |
| | dihydroxybenzene/Acid Orange 24 |
| (C.I. 20470) | Acid Black 1 (sodium salt of 1-amino-2-(4′- |
| | nitrophenylazo)-7-phenylazo-8-hydroxynaphthalene- |
| | 3,6-disulfonic acid) |
| (C.I. 23266) | (4-((4-Methylphenyl)sulfonyloxy)phenylazo)-2,2′- |
| | dimethyl-4-((2-hydroxy-5,8-disulfonato)naphthylazo)- |
| | biphenyl/Acid Red 111 |
| (C.I. 27755) | Food Black 2 |
| (C.I. 25440) | 1-(4′-Sulfonatophenylazo)-4-((2″-hydroxy-3″- |
| | acetylamino-6″,8″-disulfonato)naphthylazo)-6-sulfonato- |
| | naphthalene (tetrasodium salt)/Food Black 1 |
| (C.I. 42080) | 4-β-Hydroxyethylamino-3-nitrobenzenesulfonic acid |
| (C.I. 42090) | Acid Blue 9 |
| (C.I. 47005) | (5′,6′ or 7′)-Sulfonato-6′-methylquinoline-2,2′,Δ-1,3- |
| | indanedione/Acid Yellow 3 |
| (C.I. 14710) | Sodium salt of 4-hydroxy-3-((2-methoxyphenyl)azo)-1- |
| | naphthalenesulfonic acid/Acid Red 4 |
| | 2-Piperidino-5-nitrobenzenesulfonic acid |
| | 2-(4′-N,N-(2″-Hydroxyethyl)amino-2′- |
| | nitro)anilineethanesulfonic acid |
| | 4-β-Hydroxyethylamino-3-nitrobenzenesulfonic acid |
| | Acid Violet 49 |
| | Acid Blue 7 |
| | Acid Blue 156 |
| | Acid Blue 317 |

Most of these dyes are described in the Color Index published by The Society of Dyers and Colorists, P.O. Box 244, Perkin House, 82 Grattan Road, Bradford, Yorkshire, BD1 2JBN England.

The at least one direct dye according to the present disclosure may be present in an amount ranging from 0.001% to 20% by weight, for example from 0.005% to 10% by weight, relative to the total weight of the composition.

In the context of the present disclosure, the at least one alcohol oxidase enzyme that may be used in the dye compositions belongs to the class E.C.1.1.3 of the enzyme nomenclature (see Enzyme Nomenclature, Academic Press, Inc., 1992).

The at least one alcohol oxidase enzyme may be chosen from primary alcohol oxidases (EC1.1.3.13), secondary alcohol oxidases (EC 1.1.3.18), long-hydrocarbon-chain alcohol oxidases (EC 1.1.3.20), polyvinyl alcohol oxidases (EC 1.1.3.30), vanillyl alcohol oxidase (EC 1.1.3.38), and aromatic alcohol oxidases (EC 1.1.3.7), also known as aryl alcohol oxidases.

For example, the at least one enzyme used in the compositions according to the present disclosure may be a primary alcohol oxidase (EC 1.1.3.13).

Alcohol oxidase enzymes form a class of 2-electron oxidoreductase enzymes.

The at least one alcohol oxidase enzyme used in the dye compositions according to the present disclosure may be derived from an extract of plants, of animals, of micro-organisms, for example bacteria, fungi, yeast, microalgae or viruses, of differentiated or undifferentiated cells, obtained in vivo or in vitro, unmodified or genetically modified, or synthetic (obtained by chemical or biotechnological synthesis).

Non-limiting examples of the at least one alcohol oxidase enzyme that may be used according to the present disclosure include those extracted from the following species: Pinus, Gastropode, Manduca, Pichia, Candida, Pleurotus, Pseudomonas, Rhodococcus, Aspergillus, Kamagataella, Phanerochaete, Polyporus, Hansenula, Poria and Penicillium. For instance, the at least one alcohol oxidase enzyme may be derived from the following species: Pinus strobus, which is a plant, Gastropode mollusc and Manduca sexta, which are animals, Pichia sp. (pastoris, methanolica, angusta) and Candida sp. (boidinii, albicans, tropicalis), which are yeasts, Pleurotus pulmonarius, Aspergillus niger, Kamagataella pastoris, Phanerochaete chrysosporium, Polyporus obtusus, Hansenula polymorpha, Poria contigua, and Penicillium simplicissimum, which are fungi, and Pseudomonas pseudoalcaligenes and Rhodococcus erythropolis, which are bacteria.

In one embodiment, the alcohol oxidase enzyme used in the compositions according to the present disclosure is derived from Pichia pastoris.

The at least one alcohol oxidase enzyme may be present in the dye compositions in an amount ranging from 0.05% to 20% by weight, relative to the total weight of the composition, for example from 0.1% to 10%, such as from 0.5% to 8% by weight, relative to the weight of the dye composition.

The enzymatic activity of the at least one alcohol oxidase enzyme used in accordance with the present disclosure may be defined from the oxidation of the donor under aerobic conditions. One unit U is the amount of enzyme leading to the generation of 1 μmol of hydrogen peroxide per minute at a given pH and at a temperature of 25° C.

The at least one alcohol oxidase enzyme may be present in the dye compositions according to the present disclosure in an amount ranging from 10³U to 10⁵U, for example from 2×10³U to 5×10⁴U, per 100 g of dye composition.

The at least one substrate for the enzyme is also known as a donor for the enzyme. The nature of the at least one substrate varies as a function of the nature of the at least one alcohol oxidase enzyme that is used. The at least one substrate for the enzyme in the compositions of the present disclosure may be an alcohol chosen from branched and unbranched, saturated and unsaturated, substituted and unsubstituted primary alcohols and secondary alcohols, long-hydrocarbon-chain alcohols, and aromatic alcohols. Non-limiting examples of the at least one donor for the primary alcohol oxidases include primary alcohols comprising from 1 to 6 carbon atoms. Non-limiting examples of the at least one donor for the aryl alcohol oxidases include benzyl alcohol, 4-tert-butylbenzyl alcohol, 3-hydroxy-4-methoxybenzyl alcohol, veratryl alcohol, 4-methoxybenzyl alcohol, and cinnamyl alcohol. 2,4-hexadien-1-ol may also be used as the at least one donor for the aryl alcohol oxidases.

According to another aspect of the present disclosure, the at least one substrate for the enzyme may be a compound bearing at least one aliphatic or aromatic alcohol functional group, which is suitable for reacting with the enzyme used. The compound bearing at least one aliphatic or aromatic alcohol functional group may be, for example, an oxidation dye precursor or a cosmetically acceptable adjuvant, such as a polymer, a surfactant or a preserving agent bearing at least one alcohol functional group. As a further example, the at least one substrate for the enzyme may be an oxidation dye precursor bearing at least one aliphatic or aromatic alcohol functional group. For example, N-(β-hydroxypropyl)-para-phenylenediamine, which bears a primary alcohol functional group, may serve as an oxidation base and as a substrate for the alcohol oxidase. Similarly, oxidation couplers, such as meta- or para-aminophenol, may fulfil both functions. Such precursors are described hereinbelow. In this aspect of the present disclosure, the use of other substrates for the enzyme is optional.

Thus, the present disclosure relates to compositions for dyeing keratin fibers, for example human keratin fibers such as the hair, comprising, in a medium that is suitable for dyeing, at least one oxidation dye precursor; at least one oxidase alcohol enzyme; at least one substrate, bearing an alcohol functional group, for the enzyme; and at least one direct dye comprising an acid functional group, or a salt thereof, the at least one substrate optionally being substituted (i.e. replaced) totally or partially with the at least one oxidation dye precursor in the case where the at least one oxidation dye precursor bears at least one functional group chosen from aliphatic and aromatic alcohol functional groups.

Use of the compositions in accordance with the present disclosure may reduce the risks associated with handling hydrogen peroxide. Furthermore, the concentration of preserving agents in the compositions according to the present disclosure may be reduced by using compounds comprising at least one alcohol functional group that also have preserving properties.

The at least one substrate for the enzyme may be present in an amount ranging from 0.01% to 60% by weight, relative to the total weight of the composition, for example from 0.05% to 30% by weight, relative to the total weight of the composition.

The at least one oxidation dye precursor used in the compositions according to the present disclosure may be any art recognized oxidation base. Oxidation bases may be chosen, for example, from para-phenylenediamines, bis(phenyl)alkylenediamines, para-aminophenols, ortho-aminophenols and heterocyclic bases, and the addition salts thereof.

Among the para-phenylenediamines, non-limiting mention may be made, by way of example, of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-amino-N,N-bis(β-hydroxyethyl)-2-methylaniline, 4-amino-2-chloro-N,N-bis(β-hydroxyethyl)aniline, 2-α-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene, and the acid addition salts thereof.

Among the para-phenylenediamines mentioned above, further non-limiting examples include para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine, and the acid addition salts thereof.

Among the bis(phenyl)alkylenediamines, non-limiting mention may be made, by way of example, of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane, and the acid addition salts thereof.

Among the para-aminophenols, non-limiting mention may be made, by way of example, of para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, 4-amino-2,6-dichlorophenol, 4-amino-6[(5′-amino-2′-hydroxy-3′-methyl)phenylmethyl]-2-methylphenol, bis(5′-amino-2′-hydroxy)phenylmethane, and the acid addition salts.

Among the ortho-aminophenols, non-limiting mention may be made, by way of example, of 2-aminophenol, 2-amino-5-methylphenol, 2-amino-6-methylphenol, 5-acetamido-2-aminophenol, and the acid addition salts thereof.

Among the heterocyclic bases, non-limiting mention may be made, by way of example, of pyridine derivatives, pyrimidine derivatives, and pyrazole derivatives.

Among the pyridine derivatives, non-limiting mention may be made of the compounds described, for example, in British Patent Nos. GB 1,026,978 and GB 1,153,196, such as 2,5-diaminopyridine, 2-(4-methoxyphenyl)amino-3-aminopyridine, 3,4-diaminopyridine, and the acid addition salts thereof. Other pyridine oxidation bases that may be used in the present disclosure include, but are not limited to, the 3-aminopyrazolo[1,5-a]pyridine oxidation bases and the addition salts thereof described, for example, in French Patent Application No. FR 2,801,308. By way of example, non-limiting mention may be made of pyrazolo[1,5-a]pyrid-3-ylamine; 2-acetylaminopyrazolo[1,5-a]-pyrid-3-ylamine; 2-morpholin-4-ylpyrazolo[1,5-a]pyrid-3-ylamine; 3-aminopyrazolo[1,5-a]-pyrid ine-2-carboxyl ic acid; 2-methoxypyrazolo[1,5-a]pyrid-3-ylamine; (3-aminopyrazolo[1,5-a]pyrid-7-yl)methanol; 2-(3-aminopyrazolo[1,5-a]pyrid-5-yl)ethanol; 2-(3-aminopyrazolo[1,5-a]pyrid-7-yl)ethanol; (3-aminopyrazolo[1,5-a]pyrid-2-yl)methanol; 3,6-diaminopyrazolo[1,5-a]pyridine; 3,4-diaminopyrazolo[1,5-a]pyridine; pyrazolo[1,5-a]-pyridine-3,7-diamine; 7-morpholin-4-ylpyrazolo[1,5-a]pyrid-3-ylamine; pyrazolo[1,5-a]pyridine-3,5-diamine; 5-morpholin-4-ylpyrazolo[1,5-a]pyrid-3-ylamine; 2-[(3-aminopyrazolo[1,5-a]pyrid-5-yl)(2-hydroxyethyl)amino]ethanol; 2-[(3-aminopyrazolo[1,5-a]pyrid-7-yl)(2-hydroxyethyl)amino]ethanol; 3-aminopyrazolo[1,5-a]pyrid-5-ol; 3-aminopyrazolo[1,5-a]pyrid-4-ol; 3-aminopyrazolo[1,5-a]pyrid-6-ol; 3-aminopyrazolo[1,5-a]-pyrid-7-ol; and the acid addition salts thereof.

Among the pyrimidine derivatives, non-limiting mention may be made of the compounds described, for example, in German Patent No. DE 2,359,399; Japanese Patent Nos. JP 88-169,571 and JP 05,163,124; European Patent No. EP 0,770,375 or International Patent Publication No. WO 96/15765, such as 2-hydroxy-4,5,6-triaminopyrimidine, 2,4-dihydroxy-5,6-diaminopyrimidine, and pyrazolopyrimidine derivatives such as those mentioned in French Patent Application FR-A-2,750,048, and among which non-limiting mention may be made of pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine, 3-amino-5-methyl-7-imidazolylpropylaminopyrazolo[1,5-a]pyrimidine, the acid addition salts thereof, and the tautomeric forms thereof when a tautomeric equilibrium exists.

Among the pyrazole derivatives, non-limiting mention may be made of the compounds described in German Patent Nos. DE 3,843,892 and DE 4,133,957 and International Patent Publication Nos. WO 94/08969 and WO 94/08970, French Patent Application No. FR-A-2,733,749, and German Patent Application No. DE 19,543,988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and the acid addition salts thereof.

The at least one oxidation base may be present in an amount ranging from 0.0001% to 20% by weight, for example from 0.005% to 6% by weight, relative to the total weight of the composition.

The at least one oxidation dye precursor used in the compositions according to the present disclosure may be any art recognized oxidation coupler. Among oxidation couplers, non-limiting mention may be made of meta-phenylenediamines, meta-aminophenols, meta-diphenols, naphthalenic couplers, heterocyclic couplers, and the addition salts thereof.

Non-limiting examples that may be mentioned include 2-methyl-5-aminophenol, 5-N-(β-hydroxyethyl)amino-2-methylphenol, 6-chloro-2-methyl-5-aminophenol, 3-aminophenol, 1,3-dihydroxybenzene (or resorcinol), 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene, and the addition salts thereof.

The at least one oxidation coupler may be present in an amount ranging from 0.0001% to 20%, for example from 0.005% to 6% by weight, relative to the total weight of the composition.

The acid addition salts that may be used for the oxidation bases and couplers may be chosen, for example, from the hydrochlorides, hydrobromides, sulfates, citrates, succinates, tartrates, lactates, tosylates, benzenesulfonates, phosphates, and acetates.

The addition salts that may be used in the context of the present disclosure may be chosen, for example, from the addition salts with sodium hydroxide, potassium hydroxide, ammonia, amines and alkanolamines.

The dye compositions in accordance with the present disclosure may further comprise at least one adjuvant conventionally used in compositions for dyeing keratin fibers, such as antioxidants, penetrating agents, sequestering agents, fragrances, buffers, dispersants, surfactants, conditioners such as, for example, volatile and non-volatile, modified and unmodified silicones, cationic polymers, cations, film-forming agents, thickening polymers, ceramides, preserving agents, opacifiers, vitamins and provitamins, and direct dyes other than direct dyes comprising an acid functional group according to the present disclosure.

Each adjuvant may be present in an amount ranging from 0.01% to 20% by weight, relative to the weight of the composition.

Needless to say, the person skilled in the art will take care to select this or these optional additional compound(s) such that the beneficial properties intrinsically associated with the oxidation dye compositions in accordance with the present disclosure are not, or are not substantially, adversely affected by the envisaged addition(s).

The medium that is suitable for dyeing, also known as the dye support, may consist of water or may comprise water and at least one organic solvent to dissolve the compounds that would not be sufficiently water-soluble. As appropriate, this at least one organic solvent may be a substrate of the at least one alcohol oxidase enzyme, such as ethanol, or isopropanol. It may also be a compound other than a substrate of the at least one alcohol oxidase enzyme, chosen from, for example, polyol ethers, for instance 2-butoxyethanol, propylene glycol, propylene glycol monomethyl ether, diethylene glycol monomethyl ether, diethylene glycol monoethyl ether, and phenoxyethanol.

The at least one organic solvent may be present, for example, in an amount ranging from 1% to 40% by weight, relative to the total weight of the dye composition, for example from 5% to 30% by weight, relative to the total weight of the dye composition.

The pH of the dye compositions in accordance with the present disclosure may range from 6 to 11, for example from 7 and 10. It may be adjusted for the desired value using at least one acidifying or basifying agent usually used in the dyeing of keratin fibers, or alternatively using standard buffer systems.

Among the acidifying agents that may be used, non-limiting mention may be made, for example, of mineral and organic acids, for instance hydrochloric acid, orthophosphoric acid, sulfuric acid, carboxylic acids, such as acetic acid, tartaric acid, citric acid and lactic acid, and sulfonic acids.

Among the basifying agents that may be used, non-limiting mention may be made, for example, of aqueous ammonia, alkyl metal carbonates, alkanolamines such as monoethanolamine, diethanolamine, triethanolamine and derivatives thereof, sodium hydroxide, potassium hydroxide, and the compounds of formula (III) below: 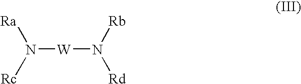
wherein:
W is chosen from propylene residues optionally substituted with a hydroxyl group or a C₁-C₄ alkyl radical; and
Ra, Rb, Rc, and Rd, which may be identical or different, are chosen from hydrogen, and C₁-C₄ alkyl and C₁-C₄ hydroxyalkyl radicals.

The dye compositions may be in various forms, such as in the form of liquids, creams or gels, or in any other form that is suitable for dyeing keratin fibers, such as human hair.

When the at least one oxidation dye and the at least one alcohol oxidase are present in the same ready-to-use composition, the composition may be, for example, free of oxygen gas, so as to avoid any premature oxidation of the at least one oxidation dye.

The present disclosure also relates to processes for dyeing keratin fibers, for example human keratin fibers, such as the hair, in which at least one dye composition according to the present disclosure is applied to these fibers, for a period of time that is sufficient to develop the desired coloration.

The color is then revealed by bringing the alcohol oxidase enzyme and its substrate into contact with atmospheric oxygen.

In one embodiment, a composition according to the present disclosure is applied to keratin fibers. After leaving it to act for a period of time ranging from 3 to 60 minutes, for example from 5 to 40 minutes, the keratin fibers are rinsed, washed with shampoo, rinsed again and then dried.

In another aspect of the present disclosure, the dye compositions may be ready-to-use compositions comprising, in a medium that is suitable for dyeing keratin fibers, at least one oxidation dye precursor, at least one alcohol oxidase enzyme, at least one substrate for the enzyme, and at least one acid direct dye, and the compositions may be stored in anaerobic form, free of oxygen gas.

According to one embodiment, the processes for dyeing keratin fibers comprise separately storing a composition (A) comprising, in a medium that is suitable for dyeing keratin fibers, at least one oxidation dye precursor, and a composition (B) comprising, in a medium that is suitable for dyeing keratin fibers, at least one alcohol oxidase enzyme, wherein composition (A) and/or composition (B) comprise at least one substrate for the enzyme, and composition (A) and/or composition (B) comprise at least one nonquinone direct dye comprising an acid functional group, and mixing together compositions (A) and (B) at the time of use before applying this mixture to keratin fibers.

According to another embodiment, the processes for dyeing keratin fibers further comprise a preliminary step that comprises separately storing a composition (A) comprising, in a medium that is suitable for dyeing keratin fibers, at least one oxidation dye precursor, at least one substrate for the enzyme, and at least one nonquinone direct dye comprising an acid functional group and a composition (B) comprising, in a medium that is suitable for dyeing keratin fibers, at least one alcohol oxidase enzyme, and mixing together compositions (A) and (B) at the time of use before applying this mixture to keratin fibers.

The color may be revealed at acidic, neutral, or alkaline pH. When the process is performed using a composition (A) comprising at least one oxidation dye precursor, at least one substrate for the enzyme, and at least one nonquinone direct dye comprising an acid functional group and a composition (B) comprising at least one alcohol oxidase enzyme, the enzyme may be added to the composition of the present disclosure just at the time of use, or it may be used starting with a composition comprising it, applied simultaneously or sequentially to the composition of the present disclosure.

Composition (B), also called the oxidizing composition, may also comprise at least one adjuvant conventionally used in compositions for dyeing keratin fibers, such as hair, and as defined above.

The pH of composition (B), also called the oxidizing composition, is such that, after mixing with dye composition (A), the pH of the resultant composition applied to the keratin fibers may range, for example, from 6 to 11, such as from 7 to 10. It may be adjusted to the desired value by means of at least one acidifying or basifying agent usually used in the dyeing of keratin fibers and as defined above.

The application of the compositions according to the present disclosure may be carried out at a temperature ranging from room temperature to 220° C., for example from room temperature to 60° C.

The present disclosure also relates to multi-compartment devices or dyeing “kits”, wherein a first compartment comprises the composition (A) as defined above and a second compartment comprises the composition (B) as defined above. These kits may be equipped with a means for applying the desired mixture to keratin fibers, such as the devices described in French Patent No. FR-2,586,913.

The present disclosure may be understood more clearly with the aid of the non-limiting examples that follow, which constitute preferred embodiments of the compositions according to the disclosure. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

### EXAMPLE

The following composition was prepared in accordance with the present disclosure.

| | | |
|---|---|---|
| Acid Black 1 (direct dye comprising an acid | 0.4 | g |
| functional group) | | |
| Ethanol (donor substrate) | 25 | g |
| para-Phenylenediamine (dye precursor) | 3 × 10⁻³ | mol |
| meta-Aminophenol (coupler) | 3 × 10⁻³ | mol |
| Alcohol oxidase | 20 000 | units |

| | |
|---|---|
| 2-Amino-2-methyl-1-propanol | qs pH 7 |

| | | |
|---|---|---|
| Distilled water | qs 100 | g |

The alcohol oxidase used is the enzyme sold by the company Biozyme Laboratories in the liquid form at a concentration of 1980 units/ml.

The unit U corresponds to the amount of enzyme leading to the generation of 1 μmol of hydrogen peroxide per minute at pH 7.5 (100 mM phosphate buffer) and at a temperature of 25° C.

The above compositions were applied to locks of natural grey permanent-waved hair containing 90% white hairs, and left to act for 30 minutes. The bath ratio was set at 5. The alcohol oxidase was added extemporaneously. The hair was then rinsed, washed with a standard shampoo, and then dried.

1. A composition for dyeing keratin fibers comprising, in a medium that is suitable for dyeing, at least one oxidation dye precursor; at least one alcohol oxidase enzyme; at least one substrate bearing an alcohol functional group for the enzyme, and at least one non-quinone direct dye comprising an acid functional group, or a salt thereof,
wherein the at least one substrate is optionally totally or partially substituted with the at least one oxidation dye precursor if the at least one oxidation dye precursor bears at least one functional group chosen from aromatic and aliphatic alcohol functional groups.

2. The composition according to claim 1, wherein the at least one non-quinone direct dye comprising an acid functional group is chosen from acidic nitro direct dyes, acidic azo dyes, acidic azine dyes, acidic triarylmethane dyes, acidic indoamine dyes, and non-quinone acidic natural dyes.

3. The composition according to claim 2, wherein the at least one non-quinone direct dye comprising an acid functional group, or a salt thereof, is chosen from the following dyes:

| | |
|---|---|
| (C.I. 10316) | Sodium salt of 2,4-dinitro-1-naphthol-7-sulfonic acid |
| (C.I. 10383) | Acid Orange 3 |
| (C.I. 13015) | Acid Yellow 9/Food Yellow 2 |
| (C.I. 14780) | Direct Red 45/Food Red 13 |
| (C.I. 13711) | Acid Black 52 |
| (C.I. 13065) | Acid Yellow 36 |
| (C.I. 14700) | Sodium salt of 1-hydroxy-2-(2′,4′-xylyl-5- |
| | sulfonatoazo)naphthalene-4-sulfonic acid/Food Red 1 |
| (C.I. 14720) | Acid Red 14/Food Red 3/Mordant Blue 79 |
| (C.I. 14805) | Acid Brown 4 |
| (C.I. 15510) | Acid Orange 7/Pigment Orange 17/Solvent Orange 49 |
| (C.I. 15985) | Food Yellow 3/Pigment Yellow 104 |
| (C.I. 16185) | Acid Red 27/Food Red 9 |
| (C.I. 16230) | Acid Orange 10/Food Orange 4 |
| (C.I. 16250) | Acid Red 44 |
| (C.I. 17200) | Acid Red 33/Food Red 12 |
| (C.I. 13683) | Sodium salt of 1-(3′-nitro-5′-sulfo-6′-oxophenylazo)-8- |
| | acetamidonaphthalene-3,6-disulfonic acid/Food Red 11 |
| (C.I. 18065) | Sodium salt of 1-hydroxy-2-(2′-methylphenylazo)-8- |
| | acetamidonaphthalene-3,6-disulfonic acid/Acid Red 35 |
| (C.I. 19125) | Acid Violet 3 |
| (C.I. 18055) | Acid Red 35 |
| (C.I. 18055) | Acid Violet 7 |
| (C.I. 18130) | Acid Red 135 |
| (C.I. 19130) | Acid Yellow 27 |
| (C.I. 19140) | Acid Yellow 23/Food Yellow 4 |
| (C.I. 20170) | 4′-(Sulfonato-2″,4″-dimethyl)bis(2,6-phenylazo)-1,3- |
| | dihydroxybenzene/Acid Orange 24 |
| (C.I. 20470) | Acid Black 1 (sodium salt of 1-amino-2-(4′- |
| | nitrophenylazo)-7-phenylazo-8-hydroxynaphthalene- |
| | 3,6-disulfonic acid) |
| (C.I. 23266) | (4-((4-Methylphenyl)sulfonyloxy)phenylazo)-2,2′- |
| | dimethyl-4-((2-hydroxy-5,8-disulfonato)naphthylazo)- |
| | biphenyl/Acid Red 111 |
| (C.I. 27755) | Food Black 2 |
| (C.I. 25440) | 1-(4′-Sulfonatophenylazo)-4-((2″-hydroxy-3″- |
| | acetylamino-6″,8″-disulfonato)naphthylazo)-6-sulfonato- |
| | naphthalene (tetrasodium salt)/Food Black 1 |
| (C.I. 42080) | 4-β-Hydroxyethylamino-3-nitrobenzenesulfonic acid |
| (C.I. 42090) | Acid Blue 9 |
| (C.I. 47005) | (5′,6′or 7′)-Sulfonato-6′-methylquinoline-2,2′,Δ-1,3- |
| | indanedione |
| (C.I. 14710) | Sodium salt of 4-hydroxy-3-((2-methoxyphenyl)azo)-1- |
| | naphthalenesulfonic acid/Acid Red 4 |
| | 2-Piperidino-5-nitrobenzenesulfonic acid |
| | 2-(4′-N N-(2″-Hydroxyethyl)amino-2′- |
| | nitro)anilineethanesulfonic acid |
| | 4-β-Hydroxyethylamino-3-nitrobenzenesulfonic acid |
| | Acid Violet 49 |
| | Acid Blue 7 |
| | Acid Blue 156 |
| | Acid Blue 317 |

4. The composition according to claim 1, wherein the at least one non-quinone direct dye comprising an acid functional group, or a salt thereof, is present in an amount ranging from 0.001% to 20% by weight, relative to the total weight of the composition.

5. The composition according to claim 1, wherein the at least one alcohol oxidase enzyme is chosen from those belonging to category E.C.1.1.3.

6. The composition according to claim 5, wherein the at least one alcohol oxidase enzyme is chosen from primary alcohol oxidases (EC1.1.3.13), secondary alcohol oxidases (EC 1.1.3.18), long-hydrocarbon-chain alcohol oxidases (EC 1.1.3.20), polyvinyl alcohol oxidases (EC 1.1.3.30), vanillyl alcohol oxidase (EC 1.1.3.38), and aromatic alcohol oxidases (EC 1.1.3.7).

7. The composition according to claim 6, wherein the at least one alcohol oxidase enzyme is derived from a species chosen from: Rhodococcus erythropolis, Pseudomonas pseudoalcaligenes, Aspergillus niger, Kamagataella pastoris, Phanerochaete chrysosporium, Polyporus obtusus, Hansenula polymorpha, Poria contigua, Penicillium simplicissimum, Pleurotus pulmonarius, Pichia sp., Candida sp., Pinus strobus, Gastropode mollusc and Manduca sexta.

8. The composition according to claim 7, wherein the at least one alcohol oxidase enzyme is derived from Pichia pastoris.

9. The composition according to claim 1, wherein the at least one alcohol oxidase enzyme is present in an amount ranging from 0.05% to 20% by weight, relative to the total weight of the composition.

10. The composition according to claim 1, wherein the at least one alcohol oxidase enzyme is present in an amount ranging from 10³U to 10⁵U, per 100 g of dye composition.

11. The composition according to claim 1, wherein the at least one substrate for the enzyme is an alcohol chosen from branched and unbranched, saturated and unsaturated, and substituted and unsubstituted primary alcohols and secondary alcohols, long-hydrocarbon-chain alcohols, and aromatic alcohols.

12. The composition according to claim 11, wherein the at least one substrate for the enzyme is present in an amount ranging from 0.01% to 60% by weight, relative to the total weight of the composition.

13. The composition according to claim 1, wherein the at least one oxidation dye precursor is an oxidation base chosen from para-phenylenediamines, bis(phenyl)alkylenediamines, para-aminophenols, ortho-aminophenols, heterocyclic bases, and the addition salts thereof.

14. The composition according to claim 13, wherein the oxidation base is present in an amount ranging from 0.0001% to 20% by weight, relative to the total weight of the composition.

15. The composition according to claim 1, wherein the at least one oxidation dye precursor is an oxidation coupler chosen from meta-phenylenediamines, meta-aminophenols, meta-diphenols, naphthalenic couplers, heterocyclic couplers, and the addition salts thereof.

16. The composition according to claim 15, wherein the oxidation coupler is present in an amount ranging from 0.0001% to 20% by weight, relative to the total weight of the composition.

17. The composition according to claim 1, further comprising at least one direct dye other than the at least one non-quinone direct dye comprising an acid functional group, or a salt thereof.

18. A process for dyeing keratin fibers, comprising
applying to the fibers at least one dye composition comprising, in a medium that is suitable for dyeing, at least one oxidation dye precursor; at least one alcohol oxidase enzyme; at least one substrate bearing an alcohol functional group for the enzyme, and at least one non-quinone direct dye comprising an acid functional group, or a salt thereof,
wherein the at least one substrate is optionally totally or partially substituted with the at least one oxidation dye precursor if the at least one oxidation dye precursor bears at least one functional group chosen from aromatic and aliphatic alcohol functional groups; and
leaving the composition on the fibers for a period that is sufficient to develop the desired coloration.

19. A process for dyeing keratin fibers, comprising
applying to the fibers a ready-to-use composition comprising, in a medium that is suitable for dyeing, at least dye composition comprising at least one oxidation dye precursor; at least one alcohol oxidase enzyme; at least one substrate bearing an alcohol functional group for the enzyme, and at least one non-quinone direct dye comprising an acid functional group, or a salt thereof,
wherein the at least one substrate is optionally totally or partially substituted with the at least one oxidation dye precursor if the at least one oxidation dye precursor bears at least one functional group chosen from aromatic and aliphatic alcohol functional groups, wherein the ready-to-use composition is stored in anaerobic form, free of oxygen gas; and
leaving the composition on the fibers for a period of time that is sufficient to develop the desired coloration.

20. The process according to claim 19, further comprising a preliminary step comprising
separately storing a composition (A) comprising, in a medium that is suitable for dyeing keratin fibers, at least one oxidation dye precursor, and a composition (B) comprising, in a medium that is suitable for dyeing keratin fibers, at least one alcohol oxidase enzyme, wherein the composition (A) and/or the composition (B) comprise at least one substrate for the at least one alcohol oxidase enzyme and the composition (A) and/or the composition (B) comprise at least one non-quinone direct dye comprising an acid functional group, or a salt thereof; and
mixing together compositions (A) and (B) at the time of use before applying this mixture to the keratin fibers.

21. The process according to claim 20, wherein the preliminary step comprises
separately storing a composition (A) comprising, in a medium that is suitable for dyeing keratin fibers, at least one oxidation dye precursor, at least one substrate for the at least one alcohol oxidase enzyme, and at least one non-quinone direct dye comprising an acid functional group, or a salt thereof and a composition (B) comprising, in a medium that is suitable for dyeing keratin fibers, at least one alcohol oxidase enzyme; and
mixing together compositions (A) and (B) at the time of use before applying this mixture to the keratin fibers.

22. A multi-compartment dyeing kit, comprising at least one first compartment comprising a composition (A) comprising, in a medium that is suitable for dyeing keratin fibers, at least one oxidation dye precursor and at least one second compartment comprising a composition (B) comprising, in a medium that is suitable for dyeing keratin fibers, at least one alcohol oxidase enzyme,
wherein the composition (A) and/or the composition (B) comprise at least one substrate for the at least one alcohol oxidase enzyme and the composition (A) and/or the composition (B) comprise at least one non-quinone direct dye comprising an acid functional group, or a salt thereof.

23. The multi-compartment dyeing kit according to claim 22, wherein the at least one first compartment comprises a composition (A) comprising, in a medium that is suitable for dyeing keratin fibers, at least one oxidation dye precursor, at least one substrate for the at least one alcohol oxidase enzyme, and at least one non-quinone direct dye comprising an acid functional group, or a salt thereof, and the at least one second compartment comprises a composition (B) comprising, in a medium that is suitable for dyeing keratin fibers, at least one alcohol oxidase enzyme.
